(19) United States
(12) Patent Application Publication
(10) Pub. No.: US 2005/0277185 A1
(43) Pub. Date: Dec. 15, 2005

(54) Binding assay device with reservoir

(76) Inventors: Andrew E. Levin, Wellesley, MA (US); Kurt Phinney, Hull, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/130,651

(22) Filed: May 17, 2005

Related U.S. Application Data
(60) Provisional application No. 60/571,956, filed on May 18, 2004.

(51) Int. Cl. C12M 1/34

(52) U.S. Cl. 435/287.1

(57) ABSTRACT

A binding assay device can provide an array of parallel channels with reservoirs for accommodating varying volumes of liquid samples for assay. The device generally can comprise a substantially planar base and a substantially planar top plate with open channels formed in its bottom surface. One embodiment includes a removable cassette, which forms the open channels when placed between the base and top plates. Reservoirs can be formed at the ends of the open channels with closed channels extending from the reservoirs to the top surface of the top plate. Open channels or elongated grooves or sockets can be formed in the top surface of the top plate to accommodate plug-in manifolds that can be in fluid communication with the closed channels. By allowing varying volumes of liquid samples to be placed in the reservoirs, the device can provide improved and/or optimized binding assay reactivities and/or band intensities.

### BACKGROUND

(1) Field

The disclosed methods and systems relate generally to an improved binding assay device and, more particularly, to an improved binding assay device having reservoirs for accommodating varying volumes of liquid samples in the channels of the device.

(2) Description of Relevant Art

Binding assays are routinely used to screen for and diagnose a whole host of diseases and conditions, including Lyme disease, herpes, acquired immunodeficiency syndrome, streptococcal infections, lupus and pregnancy, to name just a few. Such assays are relatively simple in theory, utilizing the binding affinity between two or more binding members to detect and/or quantify the presence of one of the members, referred to herein as the analyte. Binding members comprise a wide range of substances, including antigens, antibodies, haptens, complimentary nucleic acid sequences, ligands and receptors, with antigen-antibody binding member pairs used in immunoassays currently enjoying the most widespread use.

One common format of a binding assay involves immobilizing a binding member specific for the analyte on a paper like sheet or membrane. The membrane is then contacted with the test sample and appropriate reagents under conditions allowing binding to occur between the immobilized binding member and any analyte present in the sample, with means for detecting binding events also provided. Often a labeled second binding member which binds to the first binding member-analyte complex is added to provide a detectable signal on the membrane.

In the assay format described above, i.e. where the vehicle for the assay is a binding member immobilized on a membrane, a variety of devices and techniques have been developed for testing more than a single sample per membrane. For example, as described in U.S. Pat. No. 4,713,349, U.S. Pat. No. 4,834,946 and U.S. Pat. No. 5,100,626, a membrane is secured between two plates and samples applied in multiple plate channels. The lower plate has a smooth surface, while the upper plate contains an array of parallel channels machined into the plastic surface. The channels do not extend the complete thickness of the plate. A hole connects each end of each channel with the opposing surface of the plate. In practice, a thin, resilient cushion is placed on the lower plate and a membrane is placed directly over the cushion. The two plates are then fastened tightly together. Liquid samples may then be introduced through the ports in the upper surface of the upper plate, which connect with each channel. Such samples may comprise antibody-containing solutions and other reagents necessary to carry out associated blotting protocols.

In a typical application, antibodies are assayed by the Western Blot method. Thus, a blotting membrane bearing electrophoretically resolved antigens is mounted in the device. Test solutions containing antibodies are introduced into the channels and incubated with the membrane. Following aspiration of the antibody solutions, detection reagents are introduced which result in deposition of insoluble chromogenic substrates at sites where antibodies are bound to the membrane. The device may be rocked back and forth about its central axis, vertically or horizontally on a rocker platform or agitator during such incubation.

The volume of antibody or other solution which can be accommodated in each channel is determined by the dimensions of the channel. The addition of higher volumes results in spillage of solution out of the entry or exit ports at the surface of the upper plate, while lower volumes do not completely fill the channel. The operating volume is thus constrained by the dimensions of the channel, plus or minus a small tolerance.

It has been observed that mass action principles apply to binding assays carried out in the device. Accordingly, higher sensitivities of detection can be achieved through the use of greater volumes of antibody solution and detection reagents. In current devices, such higher sensitivities can be achieved solely by substituting a different upper plate in the device, containing channels of larger dimensions. While higher volumes of solution could thereby be accommodated in each channel, lower volumes were precluded in the same device. Thus, current devices are inflexible in their volume requirements, according to their specific channel dimensions. In the performance of antibody assays, however, both the volumes and titers of antibody and reagent solutions may vary. Antibody titers are frequently not under control of the operator, and reagent volumes may be limited.

Secondly, it has been observed that reactivity in solid phase binding assays, where one binding member is immobilized on a solid substrate, e.g. a membrane, and a second binding member is present in solution, is enhanced when the solution is agitated. This is presumably because agitation results in the redistribution of binding members in the solution, which have been depleted in the layer immediately above or adjacent to the solid phase through binding to the immobilized binding member. In present devices, the design of the channels effectively reduces the ability of the solution contained therein to mix with itself and thereby redistribute solute molecules during incubations. Even in the presence of external agitation, as by rocking on a vertical or horizontal rocking platform, the channel contents do not exhibit significant mixing.

It is thus desirable to provide a device with the flexibility to accommodate antibody and reagent solutions and/or other liquid samples of varying volume in the channels. Such an assay device is provided in accordance with the principles of the present invention.

### SUMMARY

The systems and methods disclosed herein can include a binding assay device that can provide an array of parallel channels with reservoirs for accommodating varying volumes of liquid samples for assay. The device generally can comprise a substantially planar base and a substantially planar top plate with open channels formed in its bottom surface. One embodiment includes a removable cassette, which forms the open channels when placed between the substantially planar base and substantially planar top plate. Reservoirs can be formed at the ends of the open channels with closed channels extending from the reservoirs to the top surface of the top plate. Open channels or elongated grooves or sockets can be formed in the top surface of the top plate to be in fluid communication with the closed channels. By allowing varying volumes of liquid samples to be placed in the reservoirs, the device can provide improved and/or optimized band intensities.

The closed channels can have openings at their termini for introduction of test samples into the reservoirs and channels. A resilient cushioning pad can be placed on the base plate to improve interchannel sealing. A removable manifold adapted for sealing engagement with the closed channel openings and in fluid communication with the channels, e.g., by fitting within the sockets formed in the top surface of the top plate, can facilitate simultaneous flushing of the channels. In assaying for an analyte, a membrane on which a binding member specific for the analyte is immobilized can be placed between the resilient pad and the open channels. After the plates have been fastened together to secure the membrane in place, the channels and reservoirs can be filled with appropriate volumes of test samples and assay reagents under conditions allowing binding and/or signaling events to occur. The channels can then be flushed to rinse away unbound materials. Additional reagents, indicators, or binding members can also be introduced into the channels to provide a detectable signal. The appropriate volumes for the test samples and assay reagents can be determined through controlled measurements and/or visual estimation of binding intensity for varying volumes of the test samples and assay reagents.

In one embodiment, an assay device for conducting a binding assay for a liquid sample of interest can include a base plate having a substantially planar inner surface, a cover plate having a substantially planar inner surface positioned parallel and superior to the base plate upper surface when the device is assembled, one or more elongated, open channels formed in, and opening at, the cover plate inner surface, a fill port extending from each of opposite end portion of each channel to an upper surface of the cover plate, and a reservoir formed in at least one end of one or more of the channels in fluid communication with the respective channel and the respective fill port to accommodate varying volumes of the liquid sample of interest.

The reservoir can extend from the fill port a distance along the length of the channel and can have a depth greater than the depth of the channel. A membrane can be placed in contact with the channel and a binding member for a component of the liquid of interest can be immobilized on the membrane. Fastening means can secure the base plate and the cover plate together and can include a plurality of screws sized to engage screw holes formed in the base plate and the cover plate. The reservoir can have a width at least as wide as a width of the channel, or less than the width of the channel. The reservoir can also include an enlarged portion of the respective fill port.

The channels can include an array of substantially parallel and closely spaced apart channels, the channels being narrow and shallow in depth, and the reservoir being formed in at least one of the channels. For the arrayed channels, the reservoir can extend from the fill port a distance along the length of the channel, and can have a depth greater than the depth of the channel. A membrane, on which a binding member has been immobilized, can be placed in contact with at least one of the channels. The assay device having the array of channels can include fastening means for securing the base plate and the cover plate together. The fastening means can include a plurality of screws sized to engage screw holes formed in the base plate and the cover plate.

The assay device can include elongated, narrow grooves formed in the cover plate upper surface and extending transversely of the channels and overlapping opposite end portions of the channels to provide a pair of depressed sockets. Each socket can have a base and opposite side walls and end walls, wherein the fill ports can extend to the bases of respective sockets. Manifold plugs can be formed of elongated strips that can be shaped to fit within and substantially fill each socket, with the plugs adapted to be frictionally sealed against the socket walls. The manifold plugs can have an inner face spaced from the socket base and an opening extending from the inner face to its opposite face, with said plugs being removably inserted within respective sockets. The plugs can be adapted to be inserted in respective sockets and a fluid can be flowed through one of the plug openings into its respective socket and through the fill ports in that socket for simultaneous introduction into the channels and/or simultaneous flushing of the channels by flowing a liquid out through the opposite channel fill ports and their overlapping socket and plug opening. For the manifold adapted device, a membrane can be placed in contact with at least one of the channels, wherein the membrane has a binding member for a component of the liquid of interest immobilized thereon.

In one embodiment, an assay cassette for receiving multiple assay samples can include a substantially planar base portion comprising a channel portion skirted by a flange portion. The channel portion can include an array of parallel downwardly opening base channels formed in its bottom surface, a pair of channel extensions extending upwardly from the top surface of base portion, with each channel extension containing an array of parallel closed channels corresponding to and in fluid communication with the base channels. The closed channels can terminate in channel openings for the introduction or removal of liquid assay samples. A reservoir can be formed in at least one end of one or more of the base channels in fluid communication with the base channel and the respective closed channel to accommodate varying volumes of liquid assay samples.

The reservoir can include an enlarged portion of the respective closed channel, and/or the reservoir can extend from the closed channel a distance along the length of the base channel, and can have a depth greater than the depth of the base channel. A membrane having a binding member for an analyte of interest immobilized thereon can be placed against the bottom surface of the cassette in contact with the downwardly opening base channels of the cassette. More than one binding member can be immobilized on the membrane, and the binding members can be for different analytes. The assay cassette can include a manifold for introducing liquids into and/or removing liquids from the closed channels simultaneously. The manifold can have a manifold opening leading to a manifold cavity formed therein, wherein the channel extension of the cassette is nestingly received by the manifold so as to place the manifold cavity in fluid communication with the channel openings of the cassette.

In one embodiment, an apparatus for use in blot screening solutions of antibody and the like materials by simultaneously reacting a substantial number of separated, microliter size volume samples of such material with a reactive material pattern on a paper-like membrane can include a base plate having a substantially planar inner surface, a cover plate having a substantially planar inner surface positioned parallel and superior to the base plate upper surface when the device is assembled, an array of elongated, open channels formed in, and opening at, the cover plate inner surface, fill ports extending from each opposite end portion of respective channels to an upper surface of said cover plate, and a reservoir formed in at least one end of at least one of the channels in fluid communication with the channel and the fill port to accommodate varying volumes of the samples.

The reservoir can extend a distance along the length of the channel adjacent the fill port, and the reservoir has a depth greater than the depth of the channel. Fastening means can secure the base plate and the cover plate together. The fastening means can include a plurality of screws sized to engage screw holes formed in the base plate and the cover plate. The apparatus can include elongated, narrow grooves formed in the cover plate upper surface and extending transversely of the channels and overlapping opposite end portions of the channels to provide a pair of depressed sockets, each having a base and opposite side walls and end walls, wherein the fill ports extend to the bases of respective sockets. Manifold plugs can be formed of an elongated strip shaped to fit within and substantially fill each socket, with each of the plugs adapted to be frictionally sealed against the socket walls. The plugs can have an inner face spaced from the socket base and an opening extending from the plug inner face to its opposite face, with the plugs being removably inserted within respective sockets. The plugs can be adapted to be inserted in respective sockets. A fluid can be flowed through one of the plug openings into its respective socket and through the fill ports in that socket for simultaneous introduction into the channels and/or simultaneous flushing of the channels by flowing a liquid out through the opposite fill ports and their overlapping socket and plug opening.

Other objects and advantages will become apparent hereinafter in view of the specification and drawings.

### BRIEF DESCRIPTION OF THE DRAWINGS

FIG. 1 illustrates a schematic perspective view of an assay device;

FIG. 2 schematically illustrates a cross-section of the assay device of FIG. 1 taken in the direction of arrows 2-2 of FIG. 1;

FIG. 3 schematically illustrates a cross-section of a cassette for use in an alternate assay device; and

FIG. 4 illustrates plots of band intensity versus sample volume for a number of antigen bands.

### DESCRIPTION

To provide an overall understanding, certain illustrative embodiments will now be described; however, it will be understood by one of ordinary skill in the art that the systems and methods described herein can be adapted and modified to provide systems and methods for other suitable applications and that other additions and modifications can be made without departing from the scope of the systems and methods described herein.

Unless otherwise specified, the illustrated embodiments can be understood as providing exemplary features of varying detail of certain embodiments, and therefore, unless otherwise specified, features, components, modules, and/or aspects of the illustrations can be otherwise combined, separated, interchanged, and/or rearranged without departing from the disclosed systems or methods. Additionally, the shapes and sizes of components are also exemplary and unless otherwise specified, can be altered without affecting the disclosed systems or methods.

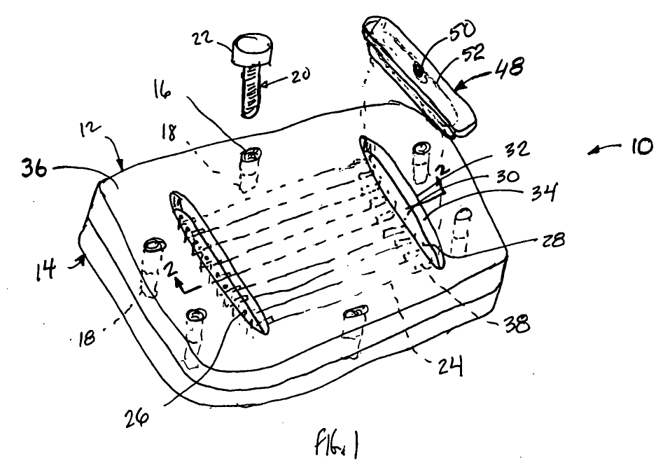

Referring to FIG. 1, an embodiment of an apparatus 10 used in screening solutions is schematically represented. Apparatus 10 can include an upper, screening plate 12 and a lower, base plate 14. For the embodiment shown, screening plate 12 can be provided with unthreaded holes 16 which can be aligned with threaded holes 18 formed in base plate 14. Screws 20, having manually graspable head 22, can be extended through the aligned holes 16, 18 for tightly fastening plates 12 and 14 together. Other means for tightly fastening plates 12 and 14 together can be contemplated, including but not limited to clamps, bolts, and/or other fastening means.

The lower surface of the screening plate 12 can be provided with numerous parallel channels 24. While the number and sizes of channels 24 can vary and can be arranged in various configurations, depending upon the intended testing purposes, channels 24 can typically be shallow, very narrow, elongated and arranged substantially parallel. For the embodiment shown in FIG. 1, channels 24 can be arranged in a single array, though other arrangements, including but not limited to pairs of spaced apart arrays can be contemplated.

Exemplary widths of channels 24 can range from 1.5 to 4.0 millimeters with their lengths ranging approximately from 5.5 to 13 centimeters. Other combinations of lengths and widths can be contemplated without change in the general design or principles of operation. Preferably, the lengths can be many times greater than the widths. As an example, channels 24 can have depths roughly in the range of 0.1 centimeters so that their volumes are in the microliter range. For example, a 1.5 millimeter wide, 5.5 centimeter long channel may have a volume of about 50 microliters, while a 4.0 millimeter width, 5.5 centimeter length channel has a volume of approximately 100 microliters. A similar 4.0 millimeter width, 13 centimeter in length channel may have a volume of close to 250 microliters. Thus, the amount of test liquid needed for filling the channels is intended to be relatively minute. The number of channels can vary considerably. For example, an array may have 16 channels that are 4.0 millimeters in width or 28 channels that are 1.5 millimeters in width, etc. and apparatus 10 can be constructed with a single channel or with fewer than 16 channels.

Each channel 24 can have an inlet hole 26 and an outlet hole 28 that can extend upwardly from locations that are near the opposite ends of channel 24. For convenience, inlet and outlet holes 26, 28 can also be referred to herein as fill ports 26, 28. For the embodiment shown in FIG. 1, holes, or fill ports 26, 28 can open into the base 30 of a groove-like socket 32. Socket 32 can be arranged transversely with respect to channels 24 at each end of the channels 24. The elongated, groove-like sockets 32 can have continuous walls 34 defining their sides and ends. In other embodiments, holes 26, 28 can open into upper surface 36 of screening plate 12, or sockets 32 can be otherwise configured.

Inlet holes 26 and outlet holes 28 can be aligned either in straight rows or in rows in which alternate holes may be slightly offset relative to each other so that they are substantially straight rows. Thus, each of the sockets 32 can overlay a row of inlet holes 26 or outlet holes 28. In use, the user can fill each of the channels 24 through one or the other of its holes 26, 28. Filling can be accomplished using a suitable commercially available automatic pipetting device or a multiple pipetting device used for handling multiple streams of microliter size quantities of liquids, though manual filling with one or more pipettes can be contemplated.

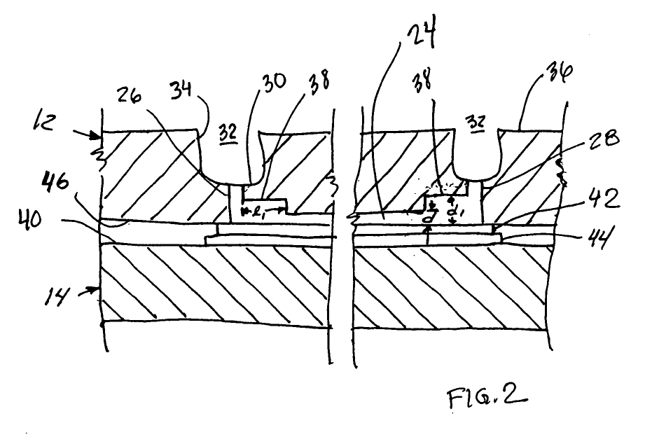

FIG. 2 schematically illustrates a cross-section of the apparatus 10 taken in the direction of arrows 2-2 of FIG. 1. One or more of channels 24 can have a reservoir 38 formed at its inlet hole 26 and/or at its outlet hole 28 to be in fluid communication with the channel 24. For the embodiment of FIGS. 1 and 2, reservoirs 38 are formed at both inlet hole 26 and outlet hole 28 of each channel 24. Reservoirs 38 can be formed by increasing one or more dimensions of channel 24 and/or holes 26, 28 to provide an increased volume of the liquid sample of interest near one or both ends of channel 24. For the embodiment shown in FIG. 2, reservoirs 38 can be formed by increasing the depth d of channel 24 to a depth d₁ over a length l₁. Other examples for forming reservoirs 38 include but are not limited to increasing a width of channel 24, increasing a width and depth, increasing a diameter of the holes 26, 28 near the channel 24, and/or combinations of the above. Thus, reservoirs 38 can provide for introducing varying volumes of the liquid sample into channels 24, as described below.

The size and/or volume of reservoirs 38 can vary among channels 24 and can be different at each end of a channel 24. However, for ease of fabrication, reservoirs 38 can be of the same size, as shown in the exemplary embodiment of FIG. 2. Exemplary volumes of reservoirs 38 can range approximately from 29 to 459 microliters, with lengths ranging approximately from 0.24 to 1.25 centimeters and depths ranging approximately from 0.064 to 1.025 centimeters. By varying the amount to which a reservoir 38 is filled, the volume of liquid sample that can be introduced into its channel 24 can be varied, without the need for varying the sizes of the reservoirs 38 or the channels 24.

The upper surface 40 of base plate 14 can act as a support for treated membrane 42. Membrane 42 can be pre-treated so as to have adsorbed or linked thereto a number of different bands of immobilized antigens, nucleic acids or other ligands, or the like. For example, membrane 42 can be treated with electrophoretically resolved polypeptides or polynucleotides of diagnostic relevance. Membrane 42 can be preferably placed upon a thin, resilient cushion sheet 44, which can be on the order of between about 0.015 to 0.040 inches in thickness, e.g., similar in thickness to a relatively thick sheet of paper. Preferably, cushion sheet 44 can be formed of a resilient, foam plastic material, such as a closed cell polyethylene foam, which can be compressed and which will tend to bulge between lines of compression. Any similar spongy or resilient material having essentially the same characteristics can be used. Preferably, the overall area of membrane 42 within a channel 24 is not affected by the presence of reservoirs 38, or by the variability of the fluid volume partially or completely filling reservoirs 38.

When screening plate 12 and base plate 14 are secured together, cushion support sheet 44 can be compressed between plate lands (not shown) of screening plate lower surface 46, which include those portions of lower surface 46 located between channels 24. Cushion sheet 44 material located between plate lands can bulge upwardly towards channels 24, causing membrane 42 to bulge into channels 24. The resilient bulging can serve to close channels 24 and tend to reduce the volume of channels 24. In addition, bulging can increase the amount of membrane 42 within each of the channels 24.

Transfer membrane 42 can be sized to overlay the array of channels 24, or at least to overlay the number of channels 24 that are to be used for any particular test. Commonly used membranes 42 can be fabricated of nonwoven paper-like sheets, such as sheets of nitrocellulose, nylon, or polyvinyldifluoridene, upon which biological analytes (e.g., proteins and nucleic acids), antigens and/or other diagnostically relevant pre-treatments have been immobilized, though other types of membranes 42 can be contemplated. Membrane 42 can be prepared through known procedures. Typically, membrane 42 can include either the same or different diagnostically relevant pre-treatments, which are applied and immobilized after prior separation by gel electrophoresis or a similar technique for fractionating biological or synthetic molecules. Alternatively, the pre-treatments can be applied to and immobilized on the membrane in separate stripes without prior electrophoretic separation. In a preferred embodiment, the pre-treatment can be a preparation of polypeptides of diagnostic relevance, e.g., for the detection of immunity to an infectious agent, such as, but not limited to, Borrelia burgdorferi, the causitive agent of Lyme disease.

In conducting a test, a cushion sheet 44, with the treated membrane 42 located upon it, can be placed upon the upper surface 40 of the base plate 14. Upper screening plate 12 can be positioned upon base plate 14 so that the array of channels 24 covers membrane 42. The two plates 12, 14 can be fastened together, for example, by manually inserting and fastening screws 20. Test solutions can be introduced through holes 26, 28 into reservoirs 38 and channels 24. Depending on the needs of the test, varying volumes of solution can be introduced by partially filling the reservoirs 38, as described previously.

After the incubation period required for the reactions between the test solutions and the membrane 42, the unbound antibody materials can be washed away, using manifolds 48, which can be inserted into sockets 32. Each manifold 48 has a central opening 50 in fluid communication with cavity 52 within manifold 48, and rinse water, or other solution, can be flowed into the central opening 50 and through cavity 52 of manifold 48 and simultaneously through the inlet holes 26 into channels 24. Similarly, the flow of water can exit from the outlet holes 28, into cavity 52 of manifold 48 inserted in socket 32 at the outlet holes 28, and then out through the central opening 50. The flow of the water can be facilitated by using a vacuum pump device, such as is typically found in laboratories, connected to the central opening 50 of a manifold 48. The manifolds 48 can be sealingly inserted into sockets 32. The liquid can be applied to the inlet manifold 48 by connecting the central opening 50 by means of a commonly available luer tubing adaptor to a length of plastic or rubber tubing, the end of which is in contact with a separate reservoir of the liquid. Liquid can be drawn from the source, through one manifold 48 and channels 24, then through the second manifold 48 to the vacuum pump. Other means of application can include, but are not limited to pouring the liquid through a small funnel or the like, or through the action of a pump producing positive pressure.

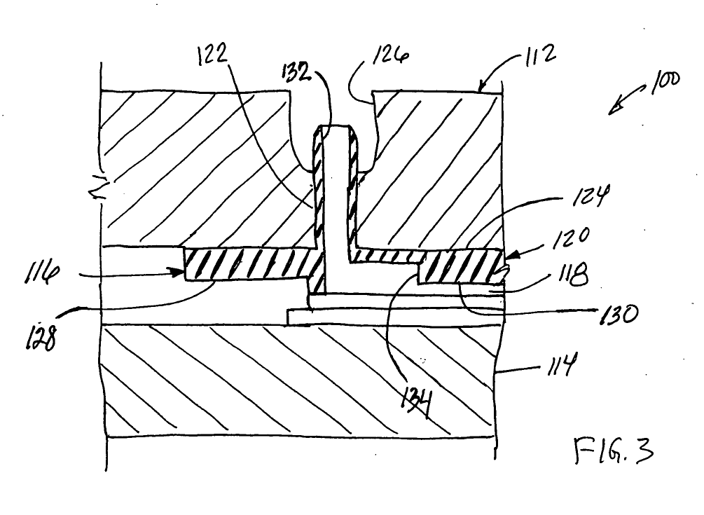

Referring to FIG. 3, there is shown a cross-section of an apparatus 100, having a cover plate 112, a base plate 114, and a cassette 116, which can form an array of parallel downwardly opening base channels 118 when inserted between cover plate 112 and base plate 114. The cross-section illustrated in FIG. 3 is taken along a length of one base channel 118. Cassette 116 generally can include a substantially planar cassette base 120 and pairs of opposing channel extensions 122 projecting perpendicularly from a top surface 124 of cassette base 120. Extensions 122 can extend through slots 126 in cover plate 112. Slots 126 can be adapted to accommodate manifolds 48 (shown in FIG. 2). Cassette base 120 can include a flange portion 128 that can skirt cassette base 120. Cassette base 120 can include an array of channels 118, formed in base bottom surface 130. Channel extensions 122 can also include an array of parallel closed channels 132 corresponding to and in fluid communication with the open base channels 118. Base channels 118 can include reservoirs 134 formed at the base of one or both of their respective closed channels 132.

As shown in FIG. 3, base channels 118, closed channels 132 and reservoirs 134 can generally function in the manner of channels 24, holes 26 and 28, and reservoirs 38 of FIG. 2, respectively. Reservoirs 134 can be formed at one or both of closed channels 132 and can be formed by increasing one or more dimensions of base channels 118 and/or closed channels 132 to provide an increased volume of the liquid sample of interest near one or both ends of base channels channel 132, generally in the manner as described for reservoirs 38 of FIG. 2.

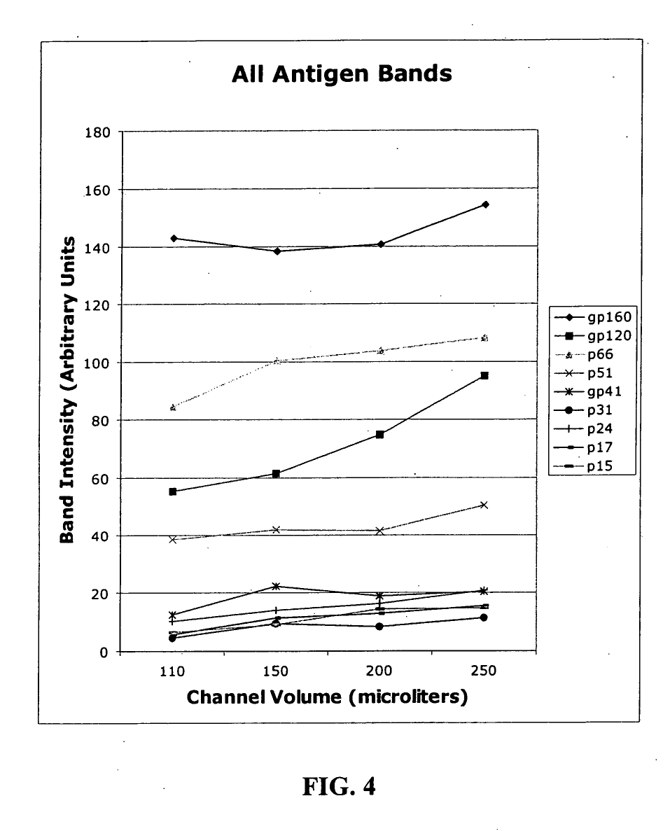

It has been found that increasing the volume of liquid samples introduced into the channels 24, 118 through the use of reservoirs 38, 136 can increase the intensity of bands in binding assays using the apparatus 10, 100 described herein. An increase in intensity can provide for improved detection of weak binding reactions and generally make the assay more easily readable. Table 1 and FIG. 4 show the band intensities, quantified by scanning, for four channels with increasing volumes of solution in an exemplary assay for HIV analytes. The channel labeled 1 represents a channel without a reservoir. Channels 2-4 include channels with reservoirs of the same size, wherein increasing volumes of solution were added into the reservoirs. As clearly indicated, intensity can generally increase with increasing reservoir volumes.

TABLE 1
Band Intensities vs. Channel Volume

Channel

| | | | |
|---|---|---|---|
| 1 | 2 | 3 | 4 |

Volume

| Antigen Band | 110 μl | 150 μl | 200 μl | 250 μl |
|---|---|---|---|---|
| gp160 | 143.05 | 138.19 | 140.53 | 154.27 |
| gp120 | 55.41 | 61.39 | 74.66 | 95.26 |
| p66 | 84.33 | 100.43 | 103.71 | 108.26 |
| p51 | 38.57 | 42.02 | 41.67 | 50.26 |
| gp41 | 12.56 | 22.27 | 18.86 | 20.4 |
| p31 | 4.65 | 9.34 | 8.45 | 11.2 |
| p24 | 10.24 | 13.84 | 16.27 | 20.81 |
| p17 | 5.84 | 11.4 | 12.76 | 15.53 |
| p15 | 6.28 | 9.27 | 14.39 | 14.77 |

In addition to the increase in band intensities, reservoirs 38, 136 can promote improved mixing of solutions in channels 24, 118. Table 2 shows migration times for three channels, each approximately 8.5 cm long, 0.239 cm wide and 0.064 cm deep. Channel 1 does not have a reservoir. The width of the reservoirs for channels 2 and 3 are the same as the width of the channel. The lengths of the reservoirs are measured from the end of the channel to the center and the depths are the measurements beyond the depths of the channels. Each channel was filled to maximum capacity, the channels were rocked and 3 ul of India ink was added into one end of each channel. The time for the India ink to migrate to the opposite end of the channel was recorded. As shown in Table 2, the migration times decreased dramatically with increasing reservoir volume.

TABLE 2
Migration Rates vs. Channel Volume

Channel

| | 1 | 2 | 3 |
|---|---|---|---|
| Reservoir Length | — | 0.24 | 1.25 |
| Reservoir Depth | — | 0.85 | 0.85 |
| Migration | 25% | 100% | 100% |
| Time (minutes) | 60 | 4 | 1 |

The slow migration shown for channel 1 can indicate that the liquid at one end of the channel cannot easily mix with liquid at the other end since the rocking of the channels serves only to move the channel contents back and forth in step. The improved mixing implied by the decreasing migration times for channels 2 and 3 can result in improved distribution of binding members throughout the solution. While the confined volume of the channel restricts mixing, the rocking of the channels can allow the liquid in the long, narrow channel to flow into the reservoir wherein mixing can occur more freely. The newly mixed liquid can then be returned to the channel as the rocking platform tilts in the opposite direction. The effect can be to regularly redistribute binding members throughout the solution, with the observed effect being greater intensity of reaction, e.g. more intense bands on the membrane, as illustrated in FIG. 4. It has been found that the effect is generally more apparent with low-titer or low-affinity antibodies than high-titer or high-affinity antibodies, as the latter may have caused the binding assay to operate in a saturated area of the dose-response curve, such that additional intensity of reaction may not be observed. Low affinity or low titer antibodies, by contrast, can generate binding reactions in a linear or logarithmic area of the dose-response curve, such that increased amounts of antibody bound translate to increased assay signal intensity. Thus, the reservoirs can serve to enhance assay signal intensity, in particular for weak signals, and so enable greater assay sensitivity.

What has thus been described includes a binding assay device that can provide an array of parallel channels with reservoirs for accommodating varying volumes of liquid samples for assay. The device generally can comprise a substantially planar base and a substantially planar top plate with open channels formed in its bottom surface. One embodiment includes a removable cassette, which forms the open channels when placed between the substantially planar base and substantially planar top plate. Reservoirs can be formed at the ends of the open channels with closed channels extending from the reservoirs to the top surface of the top plate. Open channels or elongated grooves or sockets can be formed in the top surface of the top plate to be in fluid communication with the closed channels. By allowing varying volumes of liquid samples to be placed in the reservoirs, the device can provide improved and/or optimized band intensities.

The closed channels can have openings at their termini for introduction of test samples into the reservoirs and channels. A resilient cushioning pad can be placed on the base plate to improve interchannel sealing. A removable manifold adapted for sealing engagement with the closed channel openings and in fluid communication with the channels, e.g., by fitting within the sockets formed in the top surface of the top plate, can facilitate simultaneous flushing of the channels. In assaying for an analyte, a membrane on which a binding member specific for the analyte is immobilized can be placed between the resilient pad and the open channels. After the plates have been fastened together to secure the membrane in place, the channels and reservoirs can be filled with appropriate volumes of test samples and assay reagents under conditions allowing binding and/or signaling events to occur. The channels can then be flushed to rinse away unbound materials. Additional reagents, indicators, or binding members can also be introduced into the channels to provide a detectable signal. The appropriate volumes for the test samples and assay reagents can be determined through visual estimation and/or controlled measurements of binding intensity for varying volumes of the test samples and assay reagents.

Many additional changes in the details, materials, and arrangement of parts, herein described and illustrated, can be made by those skilled in the art. Accordingly, it will be understood that the following claims are not to be limited to the embodiments disclosed herein, can include practices otherwise than specifically described, and are to be interpreted as broadly as allowed under the law.

1. An assay device for conducting a binding assay for a liquid sample of interest, the device generally comprising:
a base plate having a substantially planar inner surface;
a cover plate having a substantially planar inner surface positioned parallel and superior to said base plate upper surface when the device is assembled,
at least one elongated, open channel formed in, and opening at, said cover plate inner surface;
a fill port extending from each opposite end portion of said at least one channel to an upper surface of said cover plate; and
a reservoir formed in at least one end of said at least one channel in fluid communication with said at least one channel and the fill port to accommodate varying volumes of the liquid sample of interest.

2. The assay device as defined in claim 1, wherein the reservoir extends from the fill port a distance along the length of the channel.

3. The assay device as defined in claim 2, wherein the reservoir has a depth greater than the depth of the channel.

4. The assay device as defined in claim 3, further comprising a membrane placed in contact with the channel, wherein the membrane has a binding member for a component of the liquid of interest immobilized thereon.

5. The assay device as defined in claim 3, further comprising fastening means for securing the base plate and the cover plate together.

6. The assay device as defined in claim 5, wherein the fastening means comprises a plurality of screws sized to engage screw holes formed in the base plate and the cover plate.

7. The assay device as defined in claim 2, wherein the reservoir has a width at least as wide as a width of the channel.

8. The assay device as defined in claim 2, wherein the reservoir has a width less than a width of the channel.

9. The assay device as defined in claim 1, wherein said reservoir comprises an enlarged portion of the respective fill port.

10. The assay device as defined in claim 1, wherein the at least one open channel comprises an array of substantially parallel and closely spaced apart channels, said channels being narrow and shallow in depth, said reservoir being formed in at least one of said array of channels.

11. The assay device as defined in claim 10, wherein:
the reservoir extends from the fill port a distance along the length of the channel; and
the reservoir has a depth greater than the depth of the channel.

12. The assay device as defined in claim 11, further comprising a membrane placed in contact with at least one of the channels, wherein the membrane has a binding member for a component of the liquid of interest immobilized thereon.

13. The assay device as defined in claim 11, further comprising fastening means for securing the base plate and the cover plate together.

14. The assay device as defined in claim 13, wherein the fastening means comprises a plurality of screws sized to engage screw holes formed in the base plate and the cover plate.

15. The assay device as defined in claim 11, comprising:
elongated, narrow grooves formed in the cover plate upper surface and extending transversely of the channels and overlapping opposite end portions of the channels to provide a pair of depressed sockets, each having a base and opposite side walls and end walls, wherein the fill ports extend to the bases of respective sockets;
at least one manifold plug formed of an elongated strip shaped to fit within and substantially fill each socket, with each of said plugs adapted to be frictionally sealed against the socket walls, and having an inner face spaced from the socket base and an opening extending from said plug inner face to its opposite face; with said plugs being removably inserted within respective sockets;
whereby the plugs are adapted to be inserted in respective sockets and a fluid is adapted to be flowed through one of the plug openings into its respective socket and through the fill ports in that socket for at least one of simultaneous introduction into the channels and simultaneous flushing of the channels by flowing a liquid out through the opposite fill ports and their overlapping socket and plug opening.

16. The assay device as defined in claim 15, further comprising a membrane placed in contact with at least one of the channels, wherein the membrane has a binding member for a component of the liquid of interest immobilized thereon.

17. An assay cassette for receiving multiple assay samples, the cassette generally comprising:
a substantially planar base portion comprising a channel portion skirted by a flange portion, the channel portion containing an array of parallel downwardly opening base channels formed in its bottom surface;
a pair of channel extensions extending upwardly from the top surface of base portion, each channel extension containing an array of parallel closed channels corresponding to and in fluid communication with the base channels, wherein the closed channels terminate in channel openings for the introduction or removal of liquid assay samples; and
a reservoir formed in at least one end of at least one of the base channels in fluid communication with the base channel and the respective closed channel to accommodate varying volumes of liquid assay samples.

18. The assay device as defined in claim 17, wherein said reservoir comprises an enlarged portion of the respective closed channel.

19. The assay cassette as defined in claim 17, wherein:
the reservoir extends from the closed channel a distance along the length of the base channel; and
the reservoir has a depth greater than the depth of the base channel.

20. The assay cassette as defined in claim 19, further comprising a membrane having a binding member for an analyte of interest immobilized thereon placed against the bottom surface of the cassette in contact with the downwardly opening base channels of the cassette.

21. The assay cassette as defined in claim 20, wherein more than one binding member is immobilized on the membrane.

22. The assay cassette as defined in claim 21, wherein the binding members are for different analytes.

23. The assay cassette as defined in claim 19, further comprising a manifold for at least one of introducing liquids into and removing liquids from the closed channels simultaneously, the manifold having a manifold opening leading to a manifold cavity formed therein, wherein the channel extension of the cassette is nestingly received by the manifold placing the manifold cavity in fluid communication with the channel openings of the cassette.

24. An apparatus for use in blot screening solutions of antibody and the like materials by simultaneously reacting a substantial number of separated, microliter size volume samples of such material with a reactive material pattern on a paper-like membrane, comprising:
a base plate having a substantially planar inner surface;
a cover plate having a substantially planar inner surface positioned parallel and superior to said base plate upper surface when the device is assembled,
an array of elongated, open channels formed in, and opening at, said cover plate inner surface;
fill ports extending from each opposite end portion of respective channels to an upper surface of said cover plate; and
a reservoir formed in at least one end of at least one of the channels in fluid communication with said at least one channel and the fill port to accommodate varying volumes of the samples.

25. The apparatus as defined in claim 24, wherein:
the reservoir extends a distance along the length of the channel adjacent the fill port; and
the reservoir has a depth greater than the depth of the channel.

26. The apparatus as defined in claim 25, further comprising fastening means for securing the base plate and the cover plate together.

27. The apparatus as defined in claim 26, wherein the fastening means comprises a plurality of screws sized to engage screw holes formed in the base plate and the cover plate.

28. The apparatus as defined in claim 25, comprising:
elongated, narrow grooves formed in the cover plate upper surface and extending transversely of the channels and overlapping opposite end portions of the channels to provide a pair of depressed sockets, each having a base and opposite side walls and end walls, wherein the fill ports extend to the bases of respective sockets;
at least one manifold plug formed of an elongated strip shaped to fit within and substantially fill each socket, with each of said plugs adapted to be frictionally sealed against the socket walls, and having an inner face spaced from the socket base and an opening extending from said plug inner face to its opposite face; with said plugs being removably inserted within respective sockets;
whereby the plugs are adapted to be inserted in respective sockets and a fluid is adapted to be flowed through one of the plug openings into its respective socket and through the fill ports in that socket for at least one of simultaneous introduction into the channels and simultaneous flushing of the channels by flowing a liquid out through the opposite fill ports and their overlapping socket and plug opening.
